(19) United States
(12) Patent Application Publication
(10) Pub. No.: US 2026/0226417 A1
(43) Pub. Date: Aug. 6, 2026

(54) METHOD FOR INHIBITING AGING OF STEM CELLS

(71) Applicant: ENCELL CO., LTD, Seoul (KR)

(72) Inventors: Hong Bae JEON, Seoul (KR); Sun Jeong KIM, Seoul (KR); Soojin KWON, Seoul (KR); Jae Hoon SONG, Seoul (KR)

(21) Appl. No.: 19/159,165

(22) PCT Filed: Feb. 22, 2024
(86) PCT No.: PCT/KR2024/002328

§ 371 Date: Aug. 22, 2025

(30) Foreign Application Priority Data
Feb. 22, 2023 (KR) 10-2023-0023797

(51) Int. Cl. C12N 5/0775

(52) U.S. Cl. CPC C12N 5/0665; C12N 2501/999

(57) ABSTRACT

The present invention relates to: a method for inhibiting aging of stem cells, comprising a step of treating stem cells with nervonic acid; and a composition for inhibiting aging of stem cells, containing nervonic acid as an active ingredient. According to the present invention, aging of aged stem cells can be restored such that high cell proliferation capacity and stem cell properties are maintained, and thus cell therapy efficacy using mesenchymal stem cells can be improved.

### TECHNICAL FIELD

The present invention relates to a method of inhibiting senescence of stem cells, and more particularly, to a method of inhibiting senescence of stem cells including treating stem cells with nervonic acid, and a composition for inhibiting senescence of stem cells containing nervonic acid as an active ingredient.

### BACKGROUND ART

Mesenchymal stem cells are multipotent stem cells derived from various adult cells such as bone marrow, umbilical cord blood, placenta (or placental tissue cells), fat (or adipose tissue cells), etc. For example, mesenchymal stem cells derived from bone marrow are being studied extensively for development into cell therapies due to the multipotent ability to differentiate into adipose tissue, bone/cartilage tissue, and muscle tissue.

Recently, cell therapy technology using mesenchymal stem cells has begun to receive attention, and consequently, there is growing demand for the development of technology to activate mesenchymal stem cells isolated from the human body so as to be suitable for treatment. In particular, the patients, which are subject of cell therapy, are generally elderly, and mesenchymal stem cells harvested from tissue of the elderly tend to have low proliferation and differentiation abilities, resulting in low therapeutic efficacy. Therefore, technology to activate mesenchymal stem cells derived from the elderly to similar levels to mesenchymal stem cells derived from young people is required.

Mesenchymal stem cells, similar to other human primary cells, are known to undergo a rapid decline in cell division due to a senescence mechanism independent of telomere shortening during culture in vitro (Shibata, K. R. et al., Stem cells, 25; 2371-2382, 2007). This senescence phenomenon, although the mechanism has not yet been clearly identified, is mainly attributed to the accumulation of environmental stress due to long-term culture in vitro, which causes the expression and accumulation of p16 (INK4a) as a Cdk inhibitory protein and thus suppresses the activity of Cdk protein responsible for cell growth. In mesenchymal stem cells, it has been confirmed that cellular senescence is suppressed when the expression of p16 is inhibited by expressing the oncogene Bmi-1 (Zhang, X. et al. Biochemical and biophysical research communications, 351; 853-859, 2006). In addition, it has been reported that, when the mRNA expression of p21 (Cip1), p53, and p16 (INK4a) is inhibited by FGF-2 treatment during culture, growth arrest of mesenchymal stem cells in the G1 phase is suppressed (Ito, T. et al., Biochemical and biophysical research communications, 359; 108-114 2007). In addition, Korean Patent Application Publication No. 10-2009-0108141 discloses a method of inhibiting senescence of mesenchymal stem cells by transforming mesenchymal stem cells with a gene encoding a Wip1 protein.

Accordingly, the present inventors have ascertained that, when senescent mesenchymal stem cells are treated with nervonic acid, which is a gut microbiota-derived metabolite, mesenchymal stem cells having activity similar to that of young mesenchymal stem cells may be produced, thus culminating in the present invention.

### Disclosure

An object of the present invention is to provide a method of inhibiting senescence of stem cells.

Another object of the present invention is to provide a composition for inhibiting senescence of stem cells.

Still another object of the present invention is to provide a method of restoring stemness of stem cells.

Yet another object of the present invention is to provide a composition for restoring stemness of senescent stem cells.

Still yet another object of the present invention is to provide a method of restoring the therapeutic efficacy of stem cells.

Even yet another object of the present invention is to provide a composition for restoring the therapeutic efficacy of senescent stem cells.

In order to accomplish the above objects, the present invention provides a method of inhibiting senescence of stem cells, including treating stem cells with nervonic acid.

The present invention also provides a composition for inhibiting senescence of stem cells containing nervonic acid as an active ingredient.

The present invention also provides a method of restoring stemness of stem cells, including treating senescent stem cells with nervonic acid.

The present invention also provides a composition for restoring stemness of senescent stem cells containing nervonic acid as an active ingredient.

The present invention also provides a method of restoring the therapeutic efficacy of stem cells, including treating senescent stem cells with nervonic acid.

The present invention also provides a composition for restoring the therapeutic efficacy of senescent stem cells containing nervonic acid as an active ingredient.

### BRIEF DESCRIPTION OF DRAWINGS

FIG. 1a shows results of β-galactosidase staining used to observe the extent of senescence in a replicative senescence model of human umbilical cord-derived mesenchymal stem cells (P18, human umbilical cord-derived mesenchymal stem cells cultured for 18 passages) compared to a control group (P5, human umbilical cord-derived mesenchymal stem cells cultured for 5 passages).

FIG. 1b shows results of quantifying β-galactosidase staining in the replicative senescence model of human umbilical cord-derived mesenchymal stem cells compared to the control group.

FIG. 1c shows results of analyzing the doubling time of the replicative senescence model of human umbilical cord-derived mesenchymal stem cells (P18) compared to the control group using CCK (cell counting kit)-8 assay.

FIG. 1d shows results of determining nervonic acid as the most efficient candidate after selecting 13 gut microbiota-derived metabolite candidates through primary high-throughput screening (HTS) and secondary cell viability screening by treating the replicative senescence model of human umbilical cord-derived mesenchymal stem cells (P18) with a total of 485 gut microbiota-derived metabolites.

FIG. 2a Shows Results of Analyzing Cytotoxicity using CCK-8 assay after treating a replicative senescence model of human umbilical cord-derived mesenchymal stem cells (P18) and a control group (P5) with nervonic acid at various concentrations.

FIG. 2b shows results of B-galactosidase staining used to observe the extent of senescence by treatment at the concentration at which the difference in cell viability between the replicative senescence model of human umbilical cord-derived mesenchymal stem cells (P18) and the control group (P5) was the greatest;

FIG. 2c shows results of quantifying the β-galactosidase staining in FIG. 2b.

FIG. 3a shows results of analyzing cell proliferation after treating a replicative senescence model of human umbilical cord-derived mesenchymal stem cells (P18) and control cells (P5) with DMSO and nervonic acid for 6 days.

FIG. 3b shows results of observing cell morphology after treating the replicative senescence model of human umbilical cord-derived mesenchymal stem cells (P18) and the control group (P5) with DMSO and nervonic acid for 3 days.

FIG. 3c shows results of measuring and quantifying cell size after treating the replicative senescence model of human umbilical cord-derived mesenchymal stem cells (P18) and the control group (P5) with DMSO and nervonic acid for 3 days.

FIG. 4a shows results of assessing the expression of senescence markers P16 and P21 at the mRNA level through qRT-PCR after treating a replicative senescence model of human umbilical cord-derived mesenchymal stem cells (P18) with nervonic acid.

FIG. 4b shows results of assessing the expression of senescence markers P16 and P21 at the protein level through Western blotting after treating the replicative senescence model of human umbilical cord-derived mesenchymal stem cells (P18) with nervonic acid.

FIG. 4c shows results of quantifying the protein expression levels of senescence markers P16 and P21 after treating the replicative senescence model of human umbilical cord-derived mesenchymal stem cells (P18) with nervonic acid.

FIG. 4d Shows Results of Assessing the expression of SASP (senescence-associated secretory phenotype) markers at the mRNA level through qRT-PCR after treating the replicative senescence model of human umbilical cord-derived mesenchymal stem cells (P18) with nervonic acid.

FIG. 5 shows results of analyzing stemness using FACS after treating a replicative senescence model of human umbilical cord-derived mesenchymal stem cells (P18) with nervonic acid.

FIG. 6a shows results of β-galactosidase staining used to observe the extent of senescence in senescence-induced various human tissue-derived mesenchymal stem cells (AD-MSCs, BM-MSCs, UCB-MSCs, PL-MSCs) treated with nervonic acid.

FIG. 6b shows results of quantifying the β-galactosidase staining in FIG. 6a for each cell.

FIG. 7a shows results β-galactosidase staining used to observe the extent of senescence progression from P7 to P10 during serial subculture after treating control mesenchymal stem cells (P5) with DMSO or nervonic acid for 24 hours.

FIG. 7b shows results of quantifying the B-galactosidase staining in FIG. 7a for each passage.

FIG. 7c shows results Of assessing the expression of senescence markers P16 and P21 at the mRNA level through qRT-PCR after treating control mesenchymal stem cells (P5) with DMSO or nervonic acid followed by serial subculture up to P10.

FIG. 7d shows results of assessing the expression of SASP (senescence-associated secretory phenotype) markers IL-6, MMP 3, IGFBP 3, IGFBP5, and IGFBP7 at the mRNA level through qRT-PCR after treating control cells (P5) with DMSO or nervonic acid followed by serial subculture up to P10.

FIG. 7e shows results of analyzing cell doubling time using CCK (cell counting kit)-8 assay after treating control cells (P5) with DMSO or nervonic acid followed by serial subculture up to P10.

FIG. 8a shows results of assessing the extent of expression of Annexin V, an apoptosis marker, through immunohistochemical staining in muscle tissue one week after intravenous injection of each of young mesenchymal stem cells (P5), and a replicative senescence model (P18) that had been treated with nervonic acid or an equivalent amount of DMSO into a mouse model.

FIG. 8b shows results of quantifying the extent of expression of Annexin V based on immunohistochemical staining in FIG. 8a.

FIG. 8c shows results of assessing the expression of Annexin V, an apoptosis marker, in muscle tissue at the protein level through Western blotting one week after intravenous injection Of each of young mesenchymal stem cells (P5), and a replicative senescence model (P18) that had been treated with nervonic acid or an equivalent amount of DMSO into a mouse model.

FIG. 8d shows results of quantifying the protein expression level of Annexin V based on Western blotting in FIG. 8c.

FIG. 9a shows results of assessing the extent of expression of MyHC, a marker of muscle regeneration, through immunohistochemical staining in muscle tissue one week after intravenous injection of each of young mesenchymal stem cells (P5) , and a replicative senescence model (P18) that had been treated with nervonic acid or an equivalent amount of DMSO into a mouse model.

FIG. 9b shows results of quantifying the extent of expression of MyHC based on immunohistochemical staining in FIG. 9a.

FIG. 9c shows results of £ assessing the expression of MyHC, a marker of muscle regeneration, in muscle tissue at the protein level through Western blotting one week after intravenous injection of each of young mesenchymal stem cells (P5), and a replicative senescence model (P18) that had been treated with nervonic acid or an equivalent amount of DMSO into a mouse model.

FIG. 9d shows results of quantifying the protein expression level of MyHC based on Western blotting in FIG. 9c.

### MODE FOR INVENTION

Unless otherwise defined, all technical and scientific terms used herein have the same meanings as typically understood by those skilled in the art to which the present invention belongs. In general, the nomenclature used herein is well known in the art and is typical.

In the present invention, it was confirmed that the proportion of β-galactosidase positive cells as a senescence marker was increased in senescent mesenchymal stem cells compared to young mesenchymal stem cells isolated from umbilical cord blood, and when senescent mesenchymal stem cells were treated with nervonic acid, the proportion of β-galactosidase positive cells was decreased.

In addition, it was confirmed that, when senescent mesenchymal stem cells were treated with nervonic acid, the expression of senescence markers P16, P21, MMP3, IGFBP 5, and IGFBP7 was decreased, and the expression of mesenchymal stem cell-positive markers CD44, CD73, CD90, CD105, and CD166, which are indicators Of stemness, was increased compared to the nervonic acid-untreated group.

Senescent stem cells undergo hypertrophy and experience rapidly decreased proliferation, losing stem cell function thereof, which may lead to a decline in tissue homeostasis and regenerative ability. Moreover, there may occur a phenomenon in which the stem cell characteristic, multipotency, is reduced and stemness is also decreased to some extent.

Accordingly, an aspect of the present invention relates to a method of inhibiting senescence of stem cells, including treating stem cells with nervonic acid.

Nervonic acid used herein is a gut microbiota-derived metabolite. In an embodiment of the present invention, a replicative senescence stem cell model was treated with a library of 485 gut microbiota-derived metabolites to select a metabolite candidate group that inhibits the proliferation of senescent stem cells or causes death, thereby determining nervonic acid as a metabolite with excellent senescence control ability for stem cells.

In the present invention, nervonic acid may be used at a concentration of 1 to 200 μM, preferably a concentration of 40 to 200 μM, more preferably a concentration of 100 to 200 μM, much more preferably a concentration of 130 to 180 μM.

In the present invention, “senescent mesenchymal stem cells (MSCs) ” refer to cells having a passage number of 15 or more and having positive cells of 35% or more based on results of beta-galactosidase staining.

During senescence of stem cells, the cell cycle arrest-related p16-pRB pathway and p53-p21 pathway are activated, increasing the expression of proteins such as p16, p21, and p53. Also, abnormal structural changes occur, resulting in an increase in cell size and a longer morphology. When cells undergo senescence, beta-galactosidase expression of lysosome increases, thereby increasing the activity of senescence-associated (SA) beta-galactosidase. Also, the expression of SASP (senescence-associated secretory phenotype)-related proteins increases.

In the present invention, “young MSCs” refer to cells having a passage number of less than 6 and having positive cells of less than 5% based on results of beta-galactosidase staining.

Unlike senescent stem cells, young stem cells show low expression of proteins such as p16, p21, and p53 due to inactivation of the cell cycle arrest-related p16-pRB pathway and p53-p21 pathway, and also have a small cell size compared to senescent cells. In young stem cells, the phenomenon of increased beta-galactosidase expression or increased SASP-related protein expression, which is a hallmark of senescence, is not observed.

In the present invention, “WJ-MSCs” refer to mesenchymal stem cells derived from Wharton's jelly from umbilical cord blood. p The present invention also provides a composition for inhibiting senescence of stem cells containing nervonic acid as an active ingredient.

Another aspect of the present invention relates to a method of restoring stemness of stem cells, including treating senescent stem cells with nervonic acid.

Stem cells have an intracellular signaling system that enables continuous self-replication, and this system may be explained as stemness. Stemness may be assessed by selecting surface phenotypic markers expressed on the major cell population and performing flow cytometry (FACS).

In an embodiment of the present invention, FACS was performed on young WJ-MSCs, senescent WJ-MSCs, and nervonic acid-treated senescent WJ-MSCS to confirm stemness. Markers of mesenchymal stem cells were identified through the expression of CD44, CD73, CD90, CD105, and CD166, and markers of hematopoietic stem cell lineage used were CD14, CD11b, HLA-DR (MHCII), CD34, CD45, and CD19 (BD Biosciences, USA) to compare stemness. It was confirmed that stemness was decreased during the progression of senescence in mesenchymal stem cells, but stemness was restored by nervonic acid treatment.

Still another aspect of the present invention relates to a composition for inhibiting senescence of stem cells containing nervonic acid as an active ingredient.

Yet another aspect of the present invention relates to a composition for restoring stemness of senescent stem cells containing nervonic acid as an active ingredient.

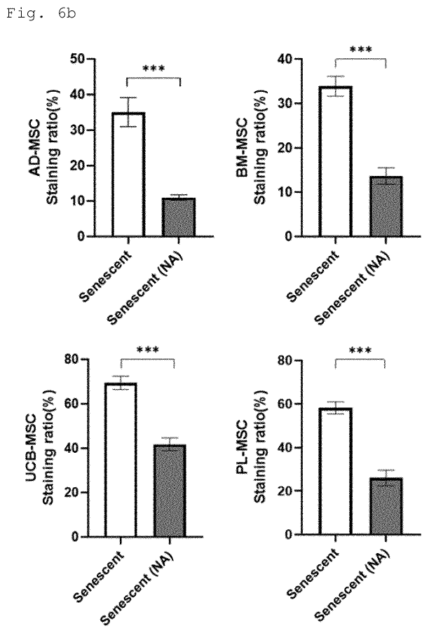

In an embodiment of the present invention, it was confirmed that, when nervonic acid was used to treat replicative senescence models of various human tissue-derived senescent mesenchymal stem cells, such as human adipose-derived mesenchymal stem cells (AD-MSCs), human bone marrow-derived mesenchymal stem cells (BM-MSCs), human umbilical cord blood-derived mesenchymal stem cells (UCB-MSCs), human placenta-derived mesenchymal stem cells (PL-MSCs), etc. the progression of senescence was significantly inhibited in all cases (FIG. 6).

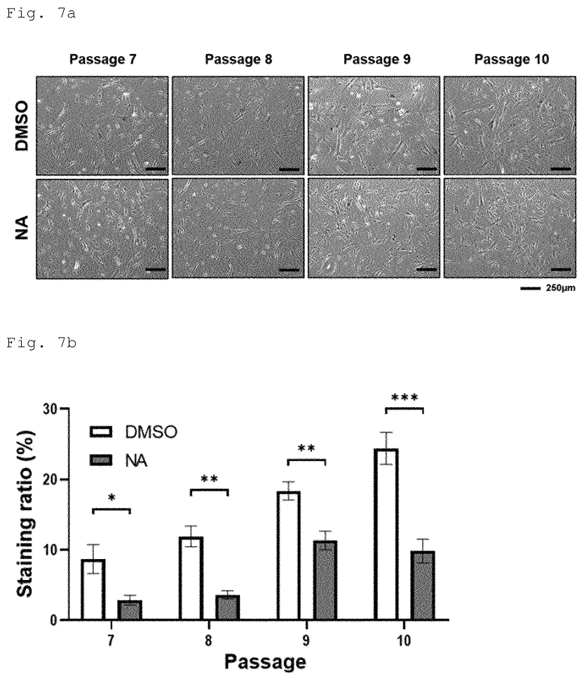

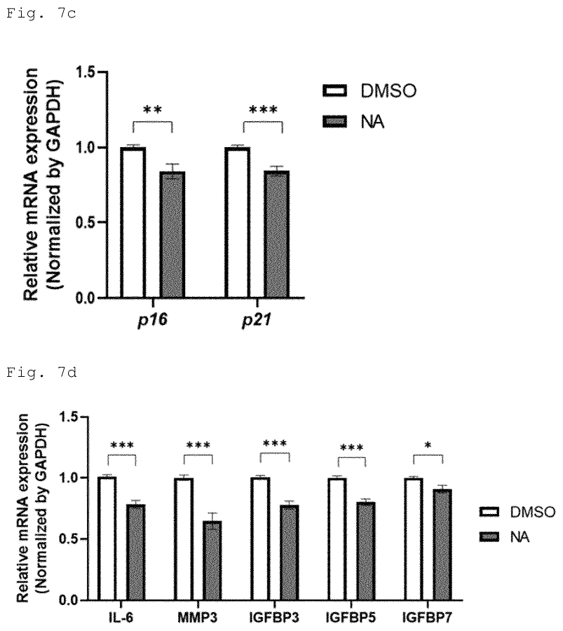

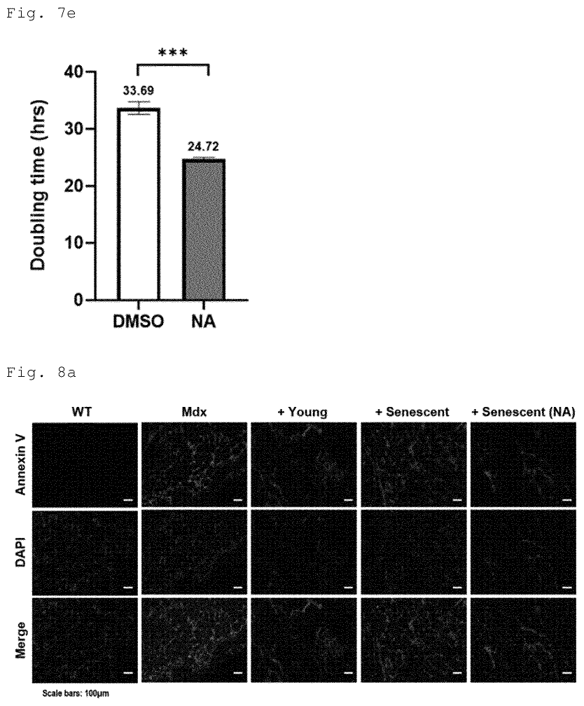

In another embodiment of the present invention, it was confirmed that the senescence of human-derived mesenchymal stem cells was delayed by continuous treatment with nervonic acid during cell culture at early passage numbers (FIG. 7).

Still yet another aspect of the present invention relates to a method of restoring the therapeutic efficacy of stem cells.

Even yet another aspect of the present invention provides a composition for restoring the therapeutic efficacy of senescent stem cells.

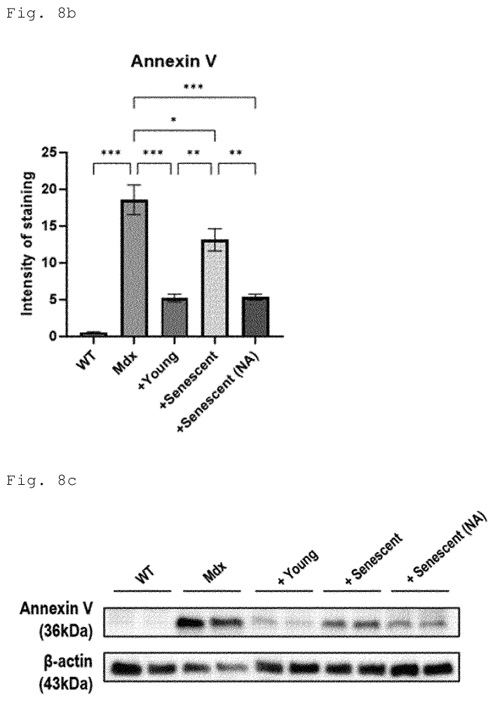

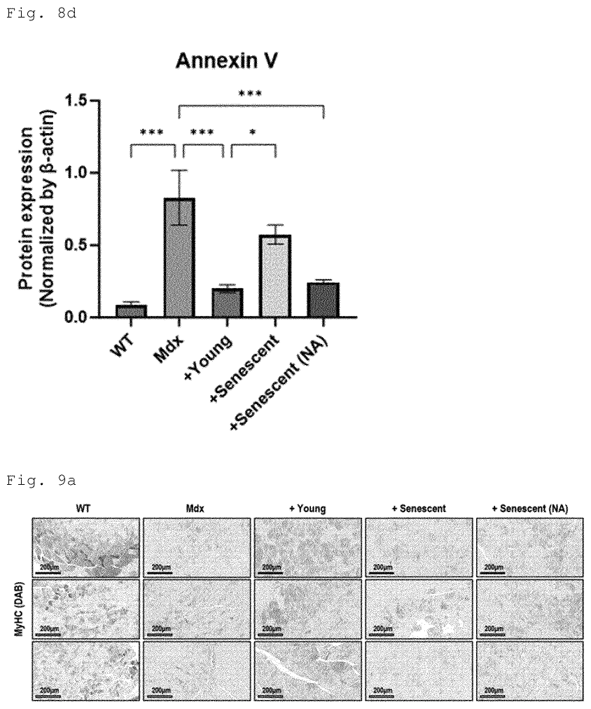

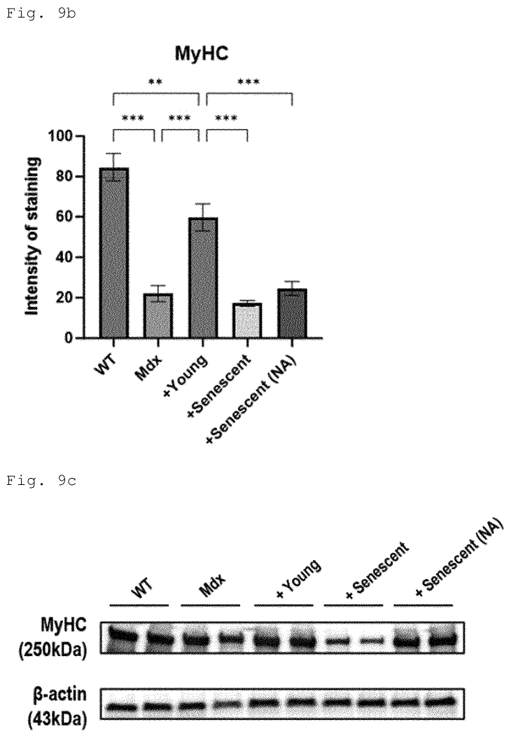

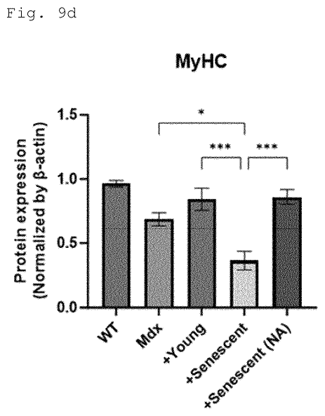

In another embodiment of the present invention, the replicative senescence model (P18) was treated with nervonic acid and then injected into a muscle disease mouse model, confirming that the therapeutic efficacy of the replicative senescence model (P18) was significantly increased by nervonic acid treatment (FIGS. 8 and 9).

Therefore, it was confirmed that nervonic acid has excellent anti-senescence efficacy in human-derived mesenchymal stem cells that have undergone replicative senescence, and may be used in a composition for controlling senescence.

### EXAMPLES

A better understanding of the present invention may be obtained through the following examples. These examples are merely set forth to illustrate the present invention and are not to be construed as limiting the scope of the present invention, as will be apparent to those skilled in the art.

Example 1. Establishment of replicative senescence model of human umbilical cord-derived mesenchymal stem cells For human umbilical cord-derived mesenchymal stem cells (Wharton's Jelly-mesenchymal stem cells, WJ-MSCs), the umbilical cord and placenta were obtained through a collaborative study with the Department of Obstetrics and Gynecology, Samsung Medical Center, in accordance with the standards of the IRB (IRB #2015-09-023-003) approved by Samsung Medical Center, and then umbilical cord-derived mesenchymal stem cells (WJ-MSCs) were isolated from the umbilical cord separated according to the method of Park et al. (Arch. Pharm. Res. 39:1171-1179, 2016). Specifically, the umbilical cord was cut into 3-4 cm lengths, the tissue was finely chopped and treated with collagenase solution (Gibco, USA) for 60-90 minutes to decompose the extracellular matrix, and then 0.25% trypsin (Gibco, USA) was added to further induce decomposition at 37° C. for 30 minutes. Thereafter, fetal bovine serum (FBS; Biowest, USA) was added, followed by centrifugation at 1,000 g for 10 minutes to obtain cells, which were then cultured in 5% CO₂ environment at 37° C. using MEM Alpha (Minimum Essential Medium, Invitrogen-Gibco, Rockville, MD) containing 10% FBS (fetal bovine serum, Invitrogen-Gibco) and 50 μg/ml gentamicin (Invitrogen-Gibco), and the resulting human umbilical cord-derived mesenchymal stem cells were used in the experiment.

In order to create a replicative senescence model of mesenchymal stem cells, the isolated cells were seeded at a density of 3×10³ cells/cm² into MEM Alpha (Minimum Essential Medium, Invitrogen-Gibco, Rockville, MD) containing 10% FBS (fetal bovine serum, Invitrogen-Gibco) and 50 μg/ml £ gentamicin (Invitrogen-Gibco) followed by serial subculture at 37° C. and 5% CO₂.

Replicative senescence was induced by subjecting human umbilical cord-derived mesenchymal stem cells (WJ-MSCs) at passage 5 to a total of 13 repeated subcultures up to passage 18. Human-derived mesenchymal stem cells at passage 18 were used as a cell model (P18) in which replicative senescence occurred, and human-derived mesenchymal stem cells at early passage (P5) in which senescence did not occur were used as control cells.

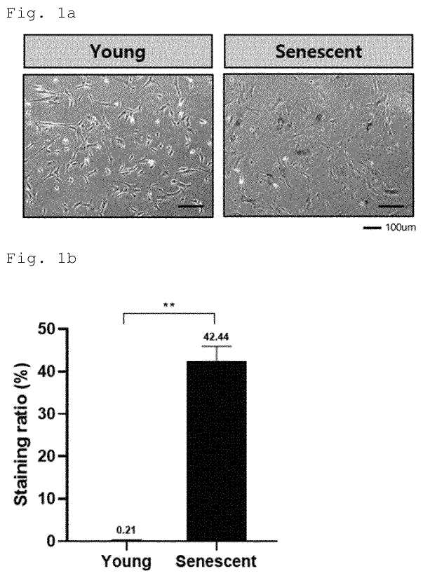

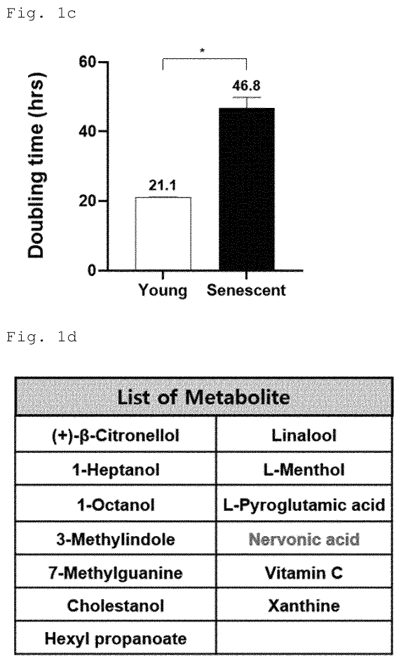

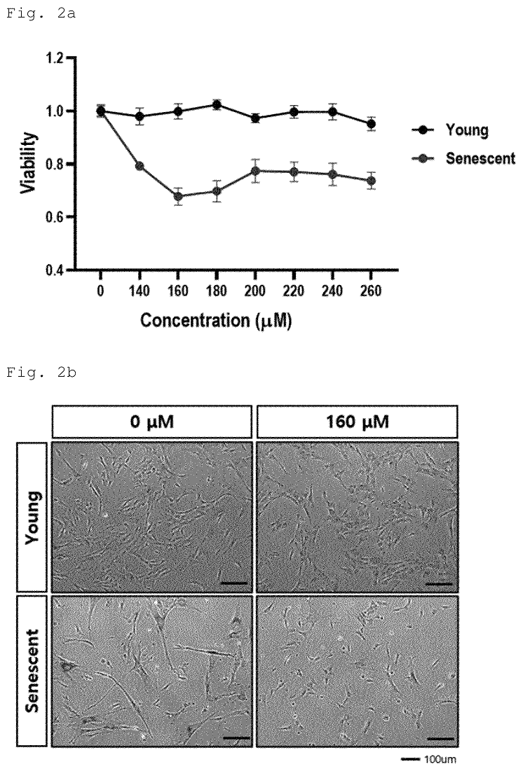

FIG. 1 shows results of comparing the extent of senescence using β-galactosidase staining to determine whether replicative senescence was induced in human umbilical cord-derived mesenchymal stem cells. Compared to the control cells (P5) in which the extent of senescence was 0.21 % of the total, the extent of senescence in the senescence-induced mesenchymal stem cells (P18) was increased to 42.44% of the total, confirming the progression of replicative senescence (FIGs. 1a and 1b). Specifically, individual cells (5×10⁴/well) were suspended in a 6-well plate and stabilized for 24 hours, followed by adding β-galactosidase staining solution and staining for 24 hours, after which the extent of senescence was measured under a microscope.

Also, the doubling time of the replicative senescence model (P18) was measured using CCK (cell counting kit)-8 assay. Thereby, the doubling time was measured to be 46.8 hours, indicating a 2.22±0.14-fold increase in the replicative senescence model (P18) compared to the control cells (P5), and was calculated based on the results collected by measurement at 0, 24, 48, and 72 hours in 96 wells (FIG. 1c).

Example 2. Screening of gut microbiota-derived metabolites for senescence control of human umbilical cord-derived mesenchymal stem cells In order to determine differences in metabolite reactivity depending on the senescence status of human umbilical cord-derived mesenchymal stem cells, a library of 485 gut microbiota-derived metabolites was used to select candidates that inhibit the proliferation or cause death of replicative senescent stem cells.

Thereby, 13 candidates showing low viability only in the replicative senescence model (P18) without affecting the viability of the control cells (P5) were selected by primary screening, and the experimental group was treated with the metabolite at 25 μM, 50 μM, 100 μM, 200 μM, 300 μM, 400 μM, and 500 μM to determine the optimal concentration of the metabolite by secondary screening, analyzing the senescence control effect at a total of 7 metabolite concentrations. Based on these results, nervonic acid, a metabolite that had the greatest effect on controlling senescence, was determined as the final candidate (FIG. 1d).

Example 3. Selection of optimal concentration of gut microbiota-derived metabolite for senescence control Nervonic acid, the gut microbiota-derived metabolite determined in Example 2, exhibited senescence control efficacy at a concentration of about 200 μM in secondary screening, so more detailed concentrations of 140 μM, 160 μM, 180 μM, 200 μM, 220 μM, 240 μM, and 260 μM were set, and then the cytotoxicity of the metabolite was measured using CCK (cell counting kit)-8 assay.

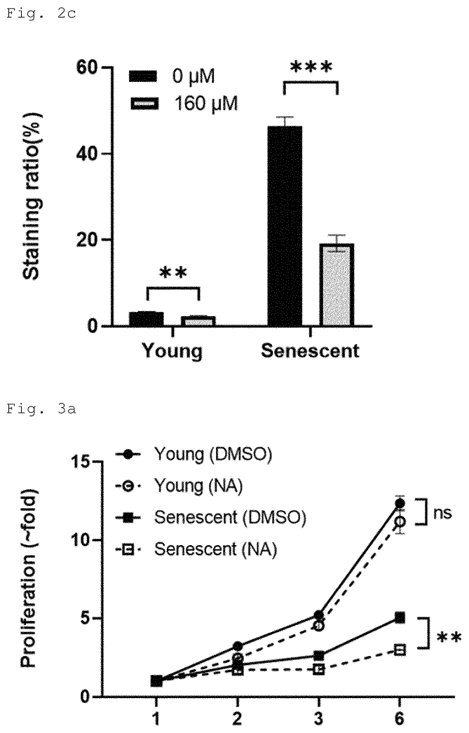

Thereby, as shown in FIG. 2a, at a nervonic acid treatment concentration of 160 μM, there was no change in cell viability in the control cells (P5), whereas a 0.68±0.03-fold decrease in cell viability in the replicative senescence model (P18) was observed compared to before treatment.

Also, based on results of measurement of the extent of senescence using β-galactosidase staining after treatment with nervonic acid (160 M), as shown in FIGS. 2b and 2c, the extent of senescence exhibited a 0.41±0.04-fold decrease compared to before treatment. Therefore, it was confirmed that this concentration was an optimal concentration that inhibited the extent of senescence by inducing apoptosis specifically in the replicative senescence model (P18) without affecting the cytotoxicity of the control cells (P5).

Example 4. Validation of gut microbiota-derived metabolite candidate for senescence control Control cells (P5) and replicative senescence-induced human umbilical cord-derived mesenchymal stem cells (P18) were treated with nervonic acid (160 μM) to analyze the effects of the gut microbiota-derived metabolite having the ability to control senescence of stem cells on the proliferation of stem cells and cell size.

Specifically, control cells (p5) and replicative senescence-induced human umbilical cord-derived mesenchymal stem cells (P18) were exposed to nervonic acid (160 μM) in culture medium, followed by culture for 6 days while replacing the medium every 3 days, and cell proliferation was assessed using CCK-8 assay.

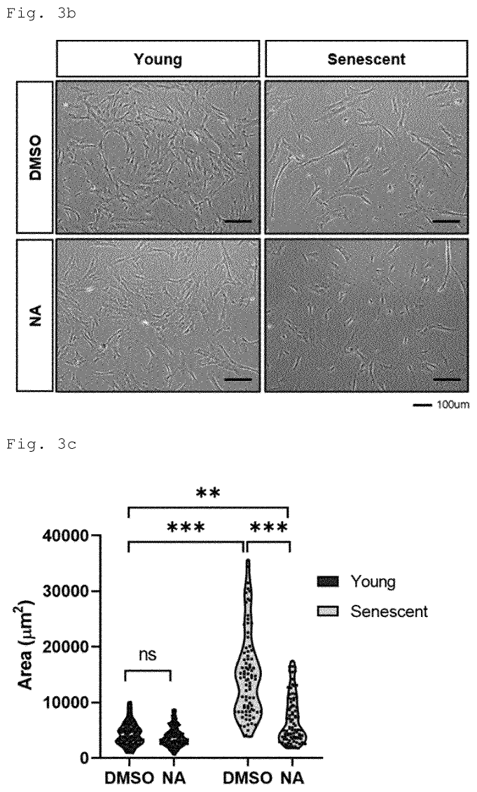

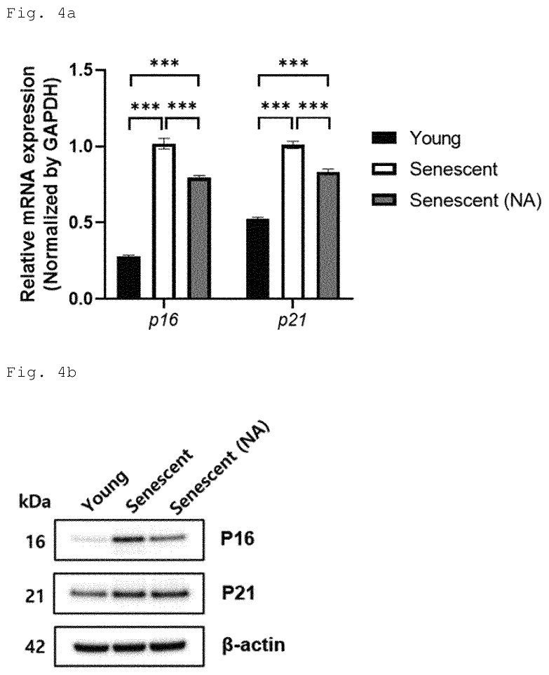

Thereby, as shown in FIG. 3a, there was no change in proliferation in the control cells (P5), and in the replicative senescence model (P18), proliferation was significantly reduced by nervonic acid (160 μM).

In addition, since cells have the characteristic of undergoing hypertrophy during the progression of cellular senescence, cell sizes were compared.

Thereby, as shown in FIGS. 3b and 3c, a 3.41±0.19-fold increase in cell size was observed in the replicative senescence model (P18) compared to the control group (P5), and treatment with nervonic acid (160 μM) led to a 0.47±0.03-fold decrease in cell size in the replicative senescence model compared to before treatment.

Example 5. Comparison of expression levels of senescence markers in gut microbiota-derived metabolite for senescence control

In order to verify the anti-senescence efficacy of nervonic acid, the expression levels of senescence-related markers in a control group (P5) and a replicative senescence model (P18) were measured after treatment with nervonic acid (160 μM).

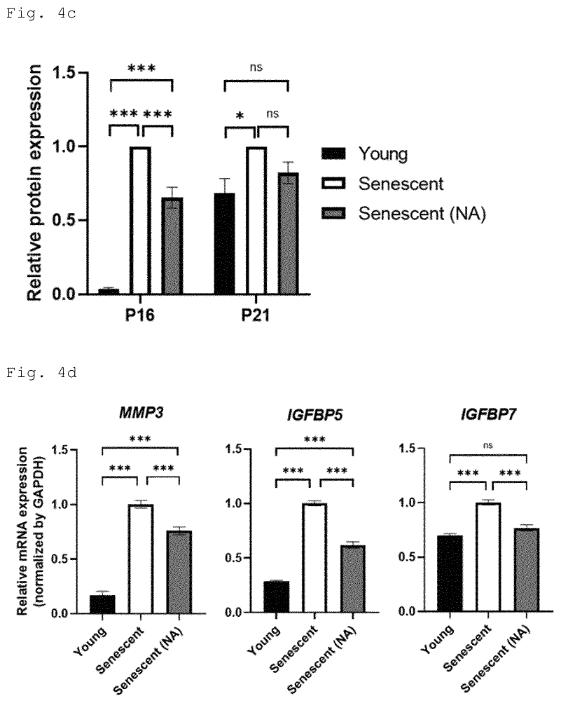

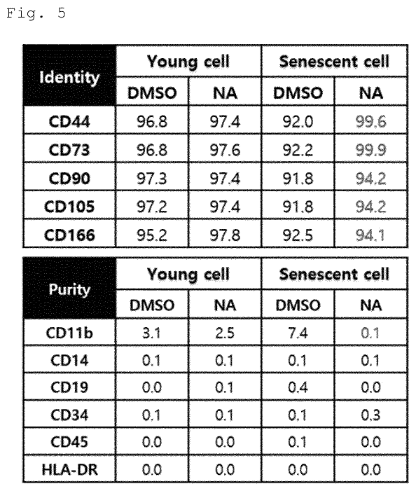

Based on results of assessment of the expression of senescence markers P16 and P21 at the mRNA level through qRT-PCR, as shown in FIG. 4a, both senescence markers P16 and P21 exhibited a 3.65±0.13-fold increase and a 1.93+0.04-fold increase, respectively, in the replicative senescence model (P18) compared to the control group (P5). After treatment with nervonic acid, respective levels of P16 and P21 significantly decreased 0.78±0.01-fold and 0.82±0.02-fold compared to before treatment.

In addition, based on results of assessment of the expression of P16 and P21 at the protein level through Western blotting, as shown in FIGS. 4b and 4c, the protein expression levels of P16 and P21 in the replicative senescence model (P18) increased 25.77-fold and 1.47-fold, respectively. After treatment with nervonic acid, however, respective expression levels of P16 and P21 decreased 0.65±0.07-fold and 0.82±0.07-fold compared to before treatment, indicating that only the expression level of P16 was significantly reduced.

Additionally, there is a report in which the gene expression of SASP (senescence-associated secretory phenotype) is increased in senescent cells, so the expression of MMP3, IGFBP5, and IGFBP7 was assessed at the mRNA level through qRT-PCR.

Thereby, as shown in FIG. 4d, it was confirmed that all of the SASP genes exhibited increased expression levels in senescent cells, and after treatment with nervonic acid, the expression significantly decreased 0.76±0.04-fold for MMP3, 0.62±0.03-fold for IGFBP 5, and 0.77±0.03-fold for IGFBP7 compared to before treatment.

Specifically, qRT-PCR performed in this example is as follows: after treating the replicative senescence model (P18) with nervonic acid for 3 days, total RNA was extracted using Trizol (Invitrogen, MA, USA) according to the method provided by the vendor. After quantifying the extracted total RNA, random hexamer and dNTP were added, and PCR was performed at 65° C. for 5 minutes. 5× SSIV buffer, 100 mM DTT, RNAse OUT, and Superscript IV RT were added and reverse transcription was performed to synthesize cDNA, and then qRT-PCR was performed to measure gene expression, and the expression levels were compared through 2^{-ΔΔCt} analysis.

Specifically, Western blotting performed in this example is as follows: cells were collected, and the cell membrane was broken using RIPA buffer, followed by centrifugation to collect the supernatant. Intracellular proteins were quantified using the Bradford assay (Bio-Rad, USA), and 20 μg of protein for each sample was loaded onto the gel, followed by electrophoresis using SDS. After transferring the protein to a polyvinylidene fluoride (PVDF) membrane, 1× TBST (Tris-buffered saline with 0.1% Tween 20) solution containing 5% skim milk (BD Difco, USA) was added and incubated at room temperature for 1 hour. Thereafter, P16 (1: 1000, Cell Signaling, USA), P21 (1: 1000, Cell Signaling, USA), and β-actin (1:5000; Santa Cruz, USA) antibodies were added and incubated overnight at 4° C. Then, the secondary antibody (anti-rabbit IgG-HRP; ABClon, Korea) was added and incubated at room temperature for one hour. Afterwards, the blots were visualized using ECL (Bio-Rad, USA) and assessed.

Example 6. Comparative analysis of stemness of gut microbiota-derived metabolite for senescence control A replicative senescence model (P18) was treated with nervonic acid (160 μM) to assess the effect thereof on stemness and adipogenesis.

The replicative senescence model (P18) was treated with nervonic acid (160 μM), and the expression changes of the mesenchymal stem cell-positive markers CD44, CD73, CD90, CD105, and CD166, as well as the mesenchymal stem cell-negative markers CD11b, CD14, CD19, CD34, CD45, and HLA-DR were confirmed through FACS.

Specifically, 3.6×10⁶ cells treated with nervonic acid (160 μM) for 3 days were centrifuged, suspended in 1.2 ml of FACS buffer (PBS 98%+FBS 2%), and allowed to stand at room temperature for 30 minutes. Thereafter, 100 ul thereof was dispensed into each of 11 1.5 ml tubes (3×10⁵ cells per tube), and a predetermined amount of antibody was added to each tube, followed by reaction at room temperature for 20 minutes in a light-blocking state. After washing by adding 200 μl of FACS buffer, centrifugation was performed at room temperature at 300 g for 5 minutes, the supernatant was removed, and the cells were suspended in 500 μl of FACS buffer and then transferred to a FACS tube for analysis.

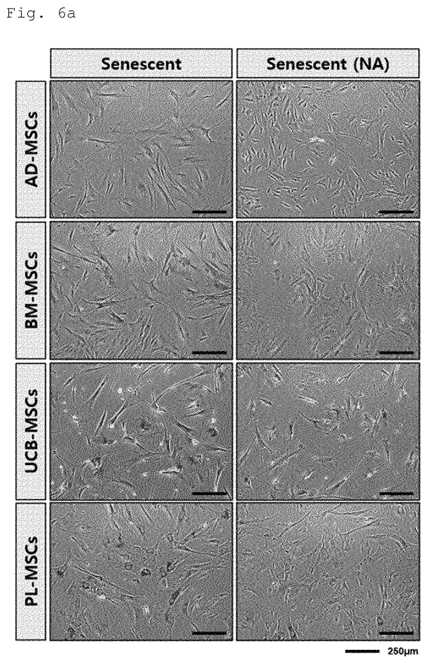

Thereby, as shown in FIG. 5, it was confirmed that the expression of mesenchymal stem cell-positive markers CD44, CD73, CD90, CD105, and CD166 was decreased in the replicative senescence model (P18) compared to the control cells (P5), but the expression of these markers was restored by nervonic acid treatment. In addition, it was confirmed that the expression of CD11b, a mesenchymal stem cell-negative marker, was increased in the replicative senescence model (P18) and then decreased by nervonic acid treatment.

Based on these results, it was confirmed that stemness was restored by nervonic acid even in senescent cells with reduced stemness.

Example 7. Confirmation of anti-senescence efficacy by treating various human tissue-derived mesenchymal stem cells with nervonic acid

Whether nervonic acid treatment exhibited the same efficacy in replicative senescence models of human adipose-derived mesenchymal stem cells (AD-MSCs), human bone marrow-derived mesenchymal stem cells (BM-MSCs), human umbilical cord blood-derived mesenchymal stem cells (UCB-MSCs), and human placenta-derived mesenchymal stem cells (PL-MSCs) was determined.

Human placenta and umbilical cord blood were harvested with maternal consent after approval from the IRB (Institutional Review Board) of Samsung Medical Center, and adipose tissue was also harvested with patient consent after approval from the IRB of Samsung Medical Center.

Human placenta-derived mesenchymal stem cells (pl-mscs) Were Isolated From Placentas Separated according to the previously reported method of Choi et al. (PLOS ONE, (2017) 12 (2): e0172642), umbilical cord blood-derived mesenchymal stem cells (UCB-MSCs) were isolated from the umbilical cord blood separated according to the method of Kim et al. (FEBS Letters, (2010) 584:3601-3608), and adipose tissue-derived mesenchymal stem cells (AD-MSCs) were isolated from adipose tissue separated according to the previously reported method of Palumbo et al. (Int. J. Mol. Sci. (2018) 19:1987).

Human bone marrow-derived mesenchymal stem cells (BM-MSCs) were purchased from Cambrex (Lonza, #PT-2501).

Each of the obtained mesenchymal stem cells was subcultured in aMEM (a-modified Minimum Essential Media) containing 10% fetal bovine serum (FBS) and 0.5% gentamicin (10 mg/ml).

The replicative senescence models of human adipose-derived mesenchymal stem cells (AD-MSCs/passage 18), human bone marrow-derived mesenchymal stem cells (BM-MSCs/passage 15), human umbilical cord blood-derived mesenchymal stem cells (UCB-MSCs/passage 16), and human placenta-derived mesenchymal stem cells (PL-MSCs/passage 18) established as described above were treated with nervonic acid for 3 days, and β-galactosidase staining was performed to observe the extent of senescence.

Thereby, as shown in FIGS. 6a and 6b, it was confirmed that the progression of senescence was significantly inhibited in various human tissue-derived senescent mesenchymal stem cells compared to the control group (DMSO).

Example 8. Confirmation Of anti-senescence efficacy by treating various human tissue-derived mesenchymal stem cells with nervonic acid Human-derived mesenchymal stem cells were treated with nervonic acid (160 μM) at each passage during cell culture from control cells (P5) to P10 to determine whether continuous treatment with nervonic acid during cell culture at early passage numbers was able to delay cellular senescence.

Specifically, from the day after human-derived mesenchymal stem cells were subcultured, treatment with 160 μM nervonic acid or an equivalent amount of DMSO (control) for 24 hours was performed, followed by replacement with the existing culture medium the next day. The above process was repeated to perform subculture up to P10 (passage 10). As such, B galactosidase staining for measuring cellular senescence was performed at P7, P8, P9, and P10, and cell doubling time and RNA extraction for qRT-PCR were both performed at P10. A more specific procedure therefor was carried out in the same manner as described above.

Thereby, as shown in FIG. 7a and FIG. 7b, it was confirmed that, when β-galactosidase staining was performed, in the control group (DMSO), the extent of senescence was increased steeply as passage progressed, whereas in the nervonic acid treatment group, the increase in the extent of senescence was significantly reduced, and the extent of senescence decreased 0.40±0.07-fold compared to the control group treated with DMSO at P10.

In addition, as shown in FIGS. 7c and 7d, when the expression of senescence markers p16 and p21 at P10 was assessed at the MRNA level, respective expression levels of p16 and p21 in the nervonic acid treatment group significantly decreased 0.84±0.05-fold and 0.84±0.03-fold compared to the control group (DMSO). Also, the expression levels of SASP markers IL-6, MMP3, IGFBP3, IGFBP5, and IGFBP7, which are highly expressed in senescent cells, significantly decreased 0.77±0.03-fold for IL-6, 0.65±0.07-fold for MMP3, 0.77±0.03-fold for IGFBP 3, 0.80±0.03-fold for IGFBP5, and 0.91±0.03-fold for IGFBP7 in the nervonic acid treatment group compared to the control group (DMSO).

Also, the doubling time of the control group (DMSO) and the nervonic acid treatment group was measured using CCK (cell counting kit)-8 assay.

Thereby, as shown in FIG. 7e, the doubling time was measured to be 24.7 hours in the replicative senescence group treated with nervonic acid, significantly shorter than in the control group (DMSO). Based on these results, it was confirmed that the senescence of human-derived mesenchymal stem cells was delayed by continuous treatment with nervonic acid at early passage numbers.

Example 9. Comparative analysis of fibrosis reduction efficacy of mesenchymal stem cells after injection various human tissue-derived mesenchymal stem cells treated with nervonic acid into muscle disease mouse model

A replicative senescence model (P18) was treated with nervonic acid (160 μM) and then injected into a muscle disease mouse model to assess the effect of nervonic acid treatment on the therapeutic efficacy of the replicative senescence model (P18) in terms of reducing apoptosis in muscle tissue.

Specifically, the muscle disease mouse model used was C57BL/10ScSn-Dmdmdx/J (mdx mouse) , and C57BL/10J with the same genetic background was used as a control group. All experiments were conducted using mice aged 4 to 5 months. The cells to be injected were human umbilical cord-derived young mesenchymal stem cells (control group) (P5) and replicative senescence model (P18). More specifically, the replicative senescence model (P18) was treated with 160 μM nervonic acid or an equivalent amount of DMSO for 72 hours during culture at P18 and then dissociated as single cells with 0.25% Trypsin/EDTA (Gibco, MT, USA), and 5×10⁴ cells per mouse were injected via the tail vein. One week after injection, both tibial muscles of the mouse model were harvested and fixed in 4% paraformaldehyde or frozen.

In order to assess the extent of apoptosis in the muscle tissue of a muscle disease mouse model after injecting mesenchymal stem cells for each condition, the extent of apoptosis in the tissue was determined through immunohistochemical staining for Annexin V, an apoptosis marker. Specifically, samples fixed as described above were embedded in paraffin and sectioned at a thickness of 4 μm. Each sample was deparaffinized and hydrated using xylene and 100% and 95% ethanol, then treated with Dako target retrieval Solution pH6 and washed three times with PBS. The washed sample was treated with Dako Real Peroxidase blocking solution for 15 minutes and washed three times with PBS, and then Annexin V (1:1000, Abcam, Cambridge, UK) antibody was added and incubated overnight at 4° C. Then, the secondary antibody (fluorescence-labeled polymer anti-rabbit) was added and incubated for 30 followed by mounting, DAPI staining, washing, and then observation and photography under a fluorescence microscope.

Thereby, as shown in FIGS. 8a and 8b, the expression level of Annexin V was significantly reduced in all the groups those injected with the young mesenchymal stem cells (P5) and those injected with the replicative senescence models (P18) treated with DMSO or nervonic acid were injected compared to the mdx control group. In particular, the decrease in expression in the injection group treated with nervonic acid was greater than that in the injection group treated with DMSO, which fell in a range statistically identical to that of the injection group with young mesenchymal stem cells.

In addition, the expression of Annexin V was assessed at the protein level. Specifically, the protein to be used in the experiment was extracted from muscle tissue. More specifically, the muscle tissue harvested one week after injection was frozen and ground using liquid nitrogen, and then added to a solution containing RIPA buffer and urea, followed by protein extraction by breaking the cell membrane using sonication. Subsequent experimental procedures were carried out in the same manner as described above, and Annexin V (1:1000, Abcam, Cambridge, UK) and B-actin (1:5000; Santa Cruz, MA, USA) primary antibodies were used.

Thereby, as shown in FIGS. 8c and 8d, the expression level was significantly reduced in the young mesenchymal stem cell (P5) injection group compared to the mdx control group, but the DMSO-treated replicative senescence model (P18) injection group did not show a significant decrease due to a reduction in efficacy caused by senescence of the mesenchymal stem cells. On the other hand, the nervonic acid treatment group showed a 0.30±0.02-fold decrease in expression compared to the mdx control group, which was a relatively significant decrease unlike the DMSO treatment group, indicating that nervonic acid is capable of partially restoring the therapeutic efficacy of the replicative senescence model to a level similar to that of the young mesenchymal stem cell (P5) injection group in terms of reducing apoptosis.

Example 10. Comparative analysis of muscle regeneration promotion efficacy after injection of various human tissue-derived mesenchymal stem cells treated with nervonic acid into muscle disease mouse model A replicative senescence model (P18) was treated with nervonic acid (160 μM) and then injected into a muscle disease mouse model to assess the effect of nervonic acid treatment on the therapeutic efficacy of the replicative senescence model (P18) in terms of muscle regeneration in muscle tissue.

In order to assess the extent of muscle regeneration in the muscle tissue of a muscle disease mouse model after injecting mesenchymal stem cells for each condition, the extent of muscle regeneration in the tissue was determined through immunohistochemical staining for MyHC (myosin heavy chain), a marker of muscle regeneration. Specifically, samples used in the experiment were harvested in the same manner as described above, and in the experimental procedures, samples retrieved in the same manner as described above were used. Retrieved and washed samples were treated with Dako Real Peroxidase blocking solution and MOM blocking solution for 15 minutes and 30 minutes, respectively, and washed three times with PBS. Then, primary antibodies against MyHC (R&D Systems, MN, USA) were added and incubated overnight at 4° C. After treatment with the primary antibodies, the secondary antibody (HRP-labeled polymer anti-mouse) was applied for 30 minutes. After washing three times with PBS, the cells were treated with Dako Lipid DAB and Substrate chromogen system, washed in the same manner as above, stained with hematoxylin, washed, dehydrated, and sealed, and then photographed and observed.

Thereby, as shown in FIGS. 9a and 9b, the expression level of MyHC was significantly increased in the injection group with the young mesenchymal stem cells (P5) compared to the mdx control group, but was not increased in the replicative senescence model (P18) injection group due to a reduction in efficacy caused by senescence of the mesenchymal stem cells. However, the expression level of MyHC was slightly increased in the injection group treated with nervonic acid compared to the injection group treated with DMSO.

In addition, the expression of MyHC was assessed at the protein level. The same experimental procedures as described above were employed, and MyHC (1:2000, Abcam, R&D Systems, MN, USA) and β-actin (1:5000; Santa Cruz, MA, USA) primary antibodies were used.

Thereby, as shown in FIGS. 9c and 9d, in the injection group with the replicative senescence model (P18) treated with DMSO, the expression level was significantly reduced compared to the mdx control group due to senescent mesenchymal stem cells. On the other hand, the expression level of MyHC was significantly increased in the injection group with the replicative senescence model (P18) treated with nervonic acid compared to the DMSO treatment group, which fell in a range statistically identical to that of the wild-type mouse and the young mesenchymal stem cell (P5) injection group.

Therefore, it was confirmed that nervonic acid has excellent anti-senescence efficacy in human-derived mesenchymal stem cells that have undergone replicative senescence, and may be used in a composition for controlling senescence.

### INDUSTRIAL APPLICABILITY

According to the present invention, senescence of senescent stem cells can be inhibited such that high cell proliferation and stemness are maintained, improving cell therapeutic efficacy using mesenchymal stem cells.

Having described certain parts of the present invention in detail above, it will be obvious to those skilled in the art that these specific descriptions are only preferred embodiments, and the scope of the present invention is not limited thereby. Accordingly, the substantial scope of the present invention will be defined by the appended claims and equivalents thereto.

1. A method of inhibiting senescence of stem cells, comprising treating stem cells with nervonic acid.

2. A composition for inhibiting senescence of stem cells containing nervonic acid as an active ingredient.

3. A method of restoring stemness of stem cells, comprising treating senescent stem cells with nervonic acid.

4. A composition for restoring stemness of senescent stem cells containing nervonic acid as an active ingredient.

5. A method of restoring therapeutic efficacy of stem cells, comprising treating senescent stem cells with nervonic acid.

6. A composition for restoring therapeutic efficacy of senescent stem cells containing nervonic acid as an active ingredient.
